(19) United States
(12) Patent Application Publication
(10) Pub. No.: US 2006/0018861 A1
(43) Pub. Date: Jan. 26, 2006

(54) Skin care composition

(76) Inventors: Minghua Chen, Kobe (JP); Peiwen Sun, Chao Yang District (CN); Akira Date, Ashiya (JP); Etsuko Yuyama, Kobe (JP); Donald Lynn Bissett, Hamilton, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/187,450

(22) Filed: Jul. 22, 2005

Related U.S. Application Data
(60) Provisional application No. 60/590,560, filed on Jul. 23, 2004.

(51) Int. Cl. A61K 8/60; A61K 31/7048

(52) U.S. Cl. 424/70.14; 514/27

(57) ABSTRACT

Disclosed is a skin care composition comprising: (1) from about 0.001% to about 10% of a flavonoid compound; (2) from about 0.01% to about 15% of a vitamin B3 compound; and (3) a dermatologically acceptable oil-in-water carrier comprising at least about 2% by weight of the entire composition of an oil component.

### CROSS REFERENCE TO RELATED APPLICATION

This application claims the benefit of U.S. Provisional Application No. 60/590,560, filed on Jul. 23, 2004.

### FIELD OF THE INVENTION

The present invention relates to a skin care composition that provides various improved skin care benefits such as skin lightening and anti-aging. The present invention particularly relates to compositions in oil-in-water form.

### BACKGROUND

Various treatment for the skin are proposed for delaying, minimizing or even eliminating skin hyperpigmentation (age spots, freckles, blotches, darkening, sallowness, uneven tone, and the like), wrinkling and other chronical changes typically associated with skin aging or environmental damage to human skin. Such treatments range from application of specialty cosmetics such as packs and masks, oral intake of vitamins, to chemical peeling, laser surgery, photofacial, and others. Generally, it is believed that effective treatment requires more time, physical, and financial commitment. There is a high desire for a treatment which is effective, but is safe and reasonably priced such that the consumer can use daily.

Various skin lightening agents and anti-aging agents are known in the art. It is also known that combination of actives may provide synergistic benefit. Flavonoids such as hesperidin are known in the art for use on the skin, for example in Japanese patent publications A11-346792, A2002-255827, A2003-137734, and U.S. Patent Application Publication 2002/13481.

For providing a skin care composition that is safe and effective for the general consumer, a combination of active agents which can be used in a variety of phase types is desired. The oil-in-water phase type is commonly used for providing compositions suitable for daily use. For example, oil-in-water phase type compositions are utilized in emulsions, moisturizing lotions, and specialty care products.

Based on the foregoing, there is a need for an oil-in-water skin care composition which provides safe and effective skin care treatment benefit over a wide range of formulations. Specifically, there is a need for a composition which provides skin lightening benefit and/or anti-aging benefit.

None of the existing art provides all of the advantages and benefits of the present invention.

### SUMMARY

The present invention is directed to a skin case composition comprising:
(1) from about 0.001% to about 10% of a flavonoid compound;
(2) from about 0.01% to about 15% of a vitamin B3 compound; and
(3) a dermatologically acceptable oil-in-water carrier comprising at least about 2% by weight of the entire composition of an oil component.

The present invention is also directed to a method of providing skin lightening benefit comprising the steps of: applying to the skin the aforementioned composition.

The present invention is also directed to a method of providing anti-aging benefit to the skin comprising the steps of: applying to the skin the aforementioned composition.

These and other features, aspects, and advantages of the present invention will become evident to those skilled in the art from a reading of the present disclosure with the appended claims.

### DETAILED DESCRIPTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

### Flavonoid Compound

The present composition comprises from about 0.001% to about 10%, preferably from about 0.01% to about 5%, more preferably from about 0.05% to about 1% of a flavonoid compound. Flavonoid compounds are known to provide antioxidant, UV absorbing, and radical scavenging benefits. Flavonoid compounds are also known to be effective in strengthening collagen structure.

Flavonoid compounds useful herein are derived from either 2-phenylbenzopyrone (I) or 3-phenylbenzopyrone (II) skeleton structure as follows. (McGraw-Hill encyclopedia of Science and technology)

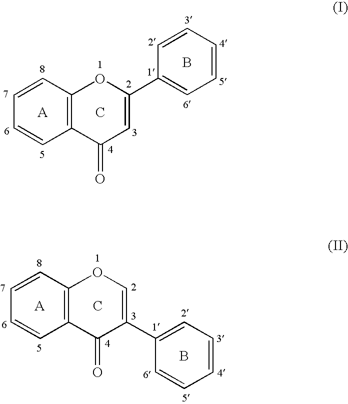

Flavonoid compounds can be further classified into different groups, depending on the oxidation level or substitution pattern of their heterocylic ring (ring C). Flavonoid compounds useful herein include unsubstituted flavanones, substituted flavanones, unsubstituted flavones, substituted flavones, unsubstituted chalcones, substituted chalcones, unsubstituted isoflavones, and substituted isoflavones. By the term “substituted” as used herein means flavonoid compounds wherein one or more hydrogen atoms of the skeleton structure as described above has been independently replaced with hydroxyl, C1-C8 alkyl, C1-C4 alkoxyl, O-glycoside, and the like or a mixture of these substituents. Flavonoid compounds particularly useful herein are selected from the group consisting of substituted flavanones, substituted flavones, substituted chalcones, substituted isoflavones, and mixtures thereof.

Flavonoid compounds can be obtained as extracts from natural sources such as plants. Examples of suitable flavonoid compounds include, but are not limited to, flavanone (unsubstituted), flavanonol (3′-hydroxy flavanone), pinocembrin (5,7-dihydroxy flavanone), pinostrobin (5-hydroxyl-7-methoxy flavanone), liquiritigenin (7,4′-dihydroxyflavanone), liquiritin (4′-glucoside-7,4′-dihydroxyflavanone), butin (7,3′,4′-trihydroxy flavanone), sakuranetin (5,4′-dihydroxy-7-methoxy flavanone), sakuranin (5-glucoside-5,4′-dihydroxy-7-methoxy flavanone), isosakuranetin (5,7-dihydroxy-4′-methoxy flavanone), poncirin (7-rhamnoglucoside-5,7-dihydroxy-4′-methoxy flavanone), naringenin (5,7,4′-trihydroxy flavanone), naringin (7-rhamnoglucoside-5,7,4′-trihydroxy flavanone), hesperitin (5,7,3′-trihydroxy-4′-methoxy flavanone), hesperidin (7-rhamnoglucoside-5,7,3′-trihydroxy-4′-methoxy flavanone), flavone (unsubstituted), chrysin (5,7-hydroxy flavone), toringin (5-glucoside-5,7-hydroxy flavone), apigenin (5,7,4′-trihydroxy flavone), apiin (7-apio-glucoside-5,7,4′-trihydroxy flavone), cosmosiin (7-glucoside-5,7,4′-trihydroxy flavone), acacetin (5,7,-dihydroxy-4′-methoxy flavone), fortunellin (7-rhamnoglucoside-5,7,-dihydroxy-4′-methoxy flavone), baicalein (5,6,7-trihydroxy flavone), baicalin (7-glucuronide-5,6,7-trihydroxy flavone), scutellarin (7-glucuronide-5,6,7,4′-tetrahydroxy flavone), diosmetin (5,7,3′-trihydroxy-4′-methoxy flavone), diosmin (7-rhamnoglucoside-5,7,3′-trihydroxy-4′-methoxy flavone), galangin (3,5,7-trihydoxy flavone), quercetin (3,5,7,3′,4′-pentahydroxy flavone), quercitrin (3-rhamnoside-3,5,7,3′,4′-pentahydroxy flavone), rutin (3-rhamnoglucoside-3,5,7,3′,4′-pentahydroxy flavone), rhamnetin (3,5,3′,4′-tetrahydroxy-7-methoxy flavone), xanthorhamnin (3-rhamnoside-3,5,3′,4′-tetrahydroxy-7-methoxy flavone), myricetin (5,7,3′,4′,5′-pentahydroxy flavonol), myricitrin (3-rhamnoside-5,7,3′,4′,5′-pentahydroxy flavonol), biflavones like fukugetin; ginkgetin and bilobetin, isoflavone, chalcone, all isomers of above substituted flavones, and mixtures thereof.

The flavonoid compounds useful herein can be synthetic materials derived or modified from naturally sourced material. With these chemical modifications, the flavonoid compounds can become more applicable to skin care compositions with improved solubility or compatibility with other composition components. Preferred modified flavonoid compounds are glycosylated, alkylated or acylated from naturally sourced material.

A particularly useful group of glycoside flavonoids herein are those selected from the general structural formula (III) 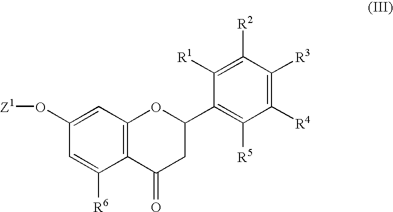
wherein R¹-R⁶ are independently selected from the group consisting of H, OH, alkoxy and hydroxyalkoxy, wherein the alkoxy or hydroxyalkoxy groups are branched or unbranched and have 1-18 carbon atoms, and wherein Z¹ is selected from the group consisting of mono- and oligoglycoside radicals. Z¹ is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

Another particularly useful group of glycoside flavonoids herein are those selected from the general structural formula (IV) 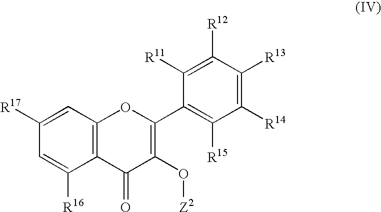
wherein R¹¹-R¹⁷ are independently selected from the group consisting of H, OH, alkoxy and hydroxyalkoxy, wherein the alkoxy or hydroxyalkoxy groups are branched or unbranched and have 1-18 carbon atoms, and wherein Z² is selected from the group consisting of mono- and oligoglycoside radicals. Z² is preferably selected from the group consisting of hexosyl radicals, more preferably rhamnosyl radicals and glucosyl radicals. It is also advantageous to use other hexosyl radicals, for example allosyl, altrosyl, galactosyl, gulosyl, idosyl, mannosyl and talosyl. It may also be advantageous according to the invention to use pentosyl radicals.

In one particularly preferred embodiment of the present invention, the glycoside flavonoid is selected from the group consisting of glucosyl hesperidin, glucosyl rutin, glucosyl myricitrin, glucosyl isoquercitrin, glucosyl quercitrin, methyl hesperidin, and mixtures thereof. These glucoside flavonoid compounds can be obtained by biochemical methods from related natural flavonoid compounds. The glucosyl group(s) can be connected to one or more hydroxides of the original substances.

A representative formula of glucosyl rutin is as follows:

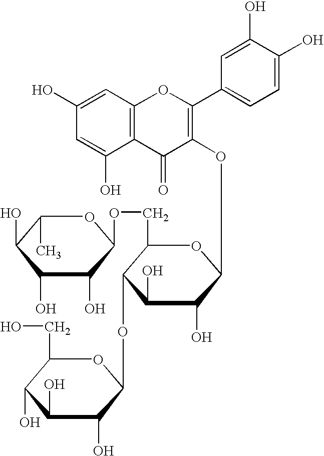

A representative formula of glucosyl hesperidin is as follows:

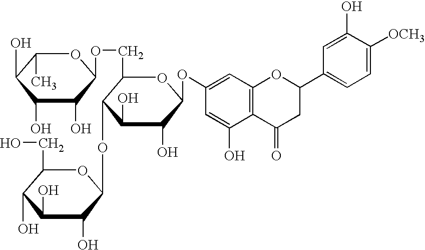

Alkylated flavonoid compounds useful herein are alkoxy or hydroxyalkoxy flavonoids that are usually derived from chemical modification of common natural flavonoids. Examples of alkylated flavonoid compounds useful herein are as follows. The formulae are merely representative, it is possible that the alkyl or hydroxyalkyl group is connected to other existing hydroxyl groups.

A representative formula of troxerutin (3′,4′,7-tri-hydroxyethoxyl-rutin) is as follows:

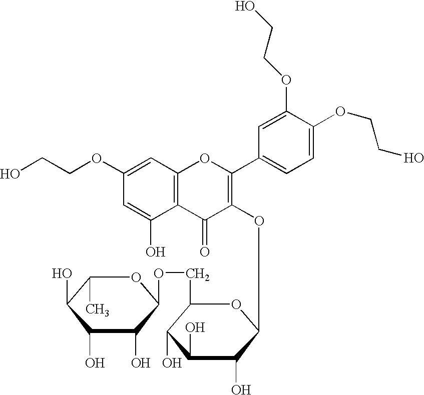

A representative formula of monoxerutin (7-hydroxyethoxyl rutin) is as follows:

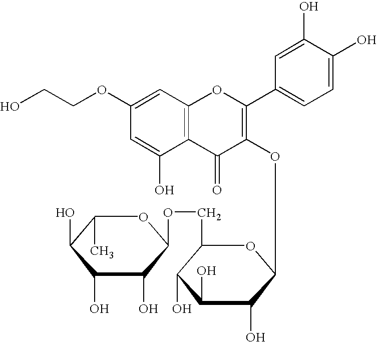

Another useful alkylated flavonoid compound, methyl hesperidin, has the general structural formula (V) 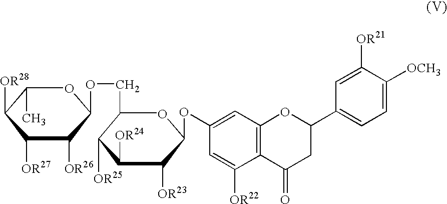
wherein R²¹-R²⁸ are independently selected from the group consisting of H or methyl, wherein at least one of R²¹-R²⁸ is methyl.

Another useful group of glycoside flavonoids herein is chalcone, which can be obtained by isomerization from any flavanone. Chalcones are highly useful in this invention due to their improved solubility which makes it easier to formulate into skin care compositions.

A representative chalcone derived from hesperidin is as follows:

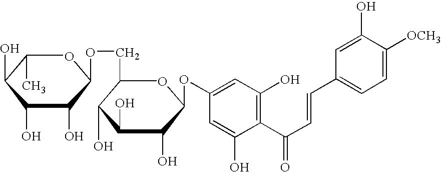

Another representative chalcone derived from hesperitin is as follows:

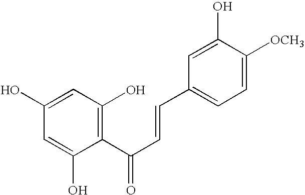

Commercially available flavonoid compounds include hesperidin, methylhesperidin, and rutin available from Alps Pharmaceutical Industry Co. Ltd. (Japan); and glucosyl hesperidin and glucosyl rutin available from Hayashibara Biochemical Laboratories, Inc. (Japan) and Toyo Sugar Refining Co. Ltd (Japan).

### Vitamin B3 Compound

The present composition comprises from about 0.01% to about 15%, preferably from about 0.1% to about 15%, more preferably from about 0.5% to about 10% of a vitamin B3 compound. Vitamin B3 compounds are known to provide, by itself, a precursor for nicotinamide adenine dinucleotide phosphate (NADP) family and its reduced form (NADPH) family of coenzymes, which enhance many metabolic enzyme reactions on the skin. Vitamin B3 compounds are also known to provide reduction in trans-epidermal water loss and excess dermal glycosaminoglycans, which are indicators for skin barrier properties.

It has been surprisingly found that, by the combined use of a flavonoid compound and a vitamin B3 compound, a composition providing synergistic skin treatment benefit over the single use of either active agent is obtained. Skin treatment benefit is particularly seen in skin lightening benefit and anti-aging benefit.

Without being bound by theory, it is believed that the flavonoid compound enhances the transportation of vitamin B3 compound. Flavonoid compounds have relatively good affinity with the cell membrane lipid bilayer, while vitamin B3 compounds have less affinity due to its generally hydrophilic structure. By the effective transportation of the 2 types of actives into the skin cells, it is believed that the 2 types of actives provide skin treatment benefits via different mechanisms in the dermis, thereby providing synergistic benefit to the skin.

Vitamin B3 compounds useful herein include, for example, those having the formula: 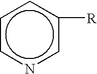
wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

### Vitamin B6 Compound

The present composition preferably further comprises from about 0.001% to about 15%, preferably from about 0.01% to about 10% by weight of the composition, more preferably from about 0.01% to about 5%, of a vitamin B6 compound.

Vitamin B6 compounds are known to provide, by itself, a coenzyme for synthesis of amino acids and nucleic acids, thereby enhancing anabolic activity, such as collagen synthesis, of skin cells. Improved collagen structure is known to provide good skin tone, and lightening appearance of the skin.

It has been surprisingly found that, by the combined use of a vitamin B6 compound in addition to a flavonoid compound and a vitamin B3 compound, a composition providing significant skin treatment benefit is obtained. Skin treatment benefit is particularly seen in skin lightening benefit and anti-aging benefit.

Without being bound by theory, it is believed that the three types of actives provide an integrated effect to the skin via different mechanisms in the dermis, thereby providing synergistic benefit to the skin.

Vitamin B6 compounds useful herein include pyridoxine; esters of pyridoxine such as pyridoxine tripahnitate, pyridoxine dipalmitate, and pyridoxine dioctanoate; amines of pyridoxine such as pyridoxamine; salts of pyridoxine such as pyridoxine HCl; and derivatives thereof such as pyridoxamine, pyridoxal, pyridoxal phosphate, and pyridoxic acid. Particularly useful vitamin, B6 compounds are selected from the group consisting of pyridoxine, esters of pyridoxine and salts of pyridoxine. The vitamin B6 compound can be synthetic or natural in origin and can be used as an essentially pure compound or mixtures of compounds (e.g., extracts from natural sources or mixtures of synthetic materials). As used herein, “vitamin B6” includes isomers and 6 tautomers of such. Commercially available vitamin B6 compound useful herein include, for example, pyridoxine HCl available from DSM, pyridoxine dipalmitate with tradename NIKKOL DP and pyridoxine dioctanoate with tradename NIKKOL DK available from Nikko Chemicals Co. Ltd.

### Oil-in-water Carrier and Composition Forms

The skin active agents of the present invention can be incorporated in various skin care compositions of oil-in-water emulsion form. The present compositions have a continuous water phase and a distinctive discontinous oil phase comprising oil component, the oil component being at least 2% by weight of the entire composition.

While the present compositions typically have a milky or turbid appearance, transparent or semi-transparent compositions can be made by adjusting the oil droplets to a size of less than about 100 nm, preferably less than about 80 nm. The size of the droplets can be determined using a Laser Scattering Particle Size Distribution Analyzer LA-910 by Horiba (Japan). The size of droplets is measured with the emulsion at a temperature of 25° C. +/− 1° C., after 30 seconds mixing. In one highly preferred embodiment, the present composition has a more or less transparent appearance having an absorbent value of less than about 2 at a wave length of 340 nm; preferably from about 1 to about 1.5 at the same wave length. Such transparent or semi-transparent emulsions are often called by the name of microemulsions, and various technology is available for achieving the desired droplet size, including PCT applications WO 00/61083 and WO 02/58665, herein incorporated by reference.

Products using the present composition include lotions, emulsions, creams, make-up bases, and liquid foundations. By incorporation of powders of certain character, the composition may comprise the character of a make-up composition.

### Oily Component

The composition of the present invention comprises oily components. Oily components useful herein can deliver skin conditioning benefits such as smoothness and softness to the skin. Oily components useful herein include, for example, fatty alcohols, silicone oils, mineral oil, petrolatum, C₇₋₄₀ straight and branched hydrocarbons such as isohexadecane, C₁₋₃₀ alcohol esters such as isopropyl isostearate, glycerides, alkylene glycol esters, propoxylated and ethoxylated derivatives, sugar ester such as sucrose polycottonseedate, vegetable oils such as coconut oil, hydrogenated vegetable oils, animal fats and oils, and C₄₋₂₀ alkyl ethers of polypropylene glycols, C₁₋₂₀ carboxylic acid esters of polypropylene glycols, and di-C₈₋₃₀ alkyl ethers. Hydrophobic nonionic surfactants, which are those being water-insoluble and having an HLB value of less than 10, can be used as oily components. Hydrophobic nonionic surfactants useful herein include, for example, cetearyl glucoside, steareth-2, laureth-4, sucrose cocate, sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

In one preferred embodiment, the oily components are selected from fatty alcohols, in that they provide skin conditioning benefits, and also in that they can form gel networks with emulsifiers which provide increased viscosity, phase stability, and conditioning benefits such as slippery feel. The fatty alcohols useful herein are a saturated, linear or branched fatty alcohol, selected from the group consisting of a saturated, linear or branched C₁₂₋₃₀ fatty alcohols, a saturated, linear or branched C₁₂₋₃₀ diols, and mixtures thereof. Preferred fatty alcohols are cetyl alcohol, stearyl alcohol, and mixtures thereof.

In another preferred embodiment, two or more oily components are selected from the group consisting of hydrocarbon oils, fatty acid esters, and silicone oils.

The oily components are included in the composition at a level by weight of, preferably from about 2% to about 50%, more preferably from about 2% to about 20%, still more preferably from about 2% to about 10%, in view of providing skin conditioning benefits such as smoothness.

### Emulsifier

The present composition preferably comprises an emulsifier for dispersing the oil components in the aqueous phase. A wide variety of emulsifiers can be employed herein. Known or conventional emulsifiers can be used in the composition, provided that the selected emulsifying agent is chemically and physically compatible with essential components of the composition, and provides the desired dispersion characteristics.

Non-limiting examples of emulsifiers useful herein are various non-ionic and anionic emulsifiers such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Nonlimiting examples of other emulsifiers for use herein include: polyethylene glycol 20 sorbitan monolaurate (polysorbate 20), polyethylene glycol 5 soya sterol, steareth-20, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, polyoxyethylene 20 sorbitan trioleate (polysorbate 85), sorbitan monolaurate, polyoxyethylene 4 lauryl ether sodium stearate, polyglyceryl-4 isostearate, hexyl laurate, PPG-2 methyl glucose ether distearate, ceteth-10, diethanolamine cetyl phosphate, glyceryl stearate, PEG 40 hydrogenated castor oil, PEG-60 hydrogenated castor oil, and mixtures thereof.

In one highly preferred embodiment, the oil-in-water composition of the present invention provides a microemulsion which is transparent or translucent in appearance. The microemulsion can be formed via selection of surfactants. Hereinafter, the surfactant selection for providing a microemulsion is referred to as the “first surfactant system”. Preferably, the first surfactant system is liquid at 40° C., more preferably at 25° C.

The first surfactant system comprises one or more nonionic surfactants selected from the group consisting of polysorbates, polyoxyalkylene hydrogenated caster oils, polyglycerin alkyl esters having the C10-20 of alkylsubstitute, polyoxyethylene sterols, and polyoxyethylene hydrogenated sterols. The hydrophilic-lipophilic balance (HLB) of the first surfactant system as a whole is 10 or more, preferably about 12 or more, more preferably from about 14 to about 20. Preferably, the first surfactant system consists essentially of nonionic surfactants having an HLB of 10 or more, more preferably 12 or more, still more preferably from about 14 to about 20.

Polyoxyalkylene hydrogenated castor oils useful herein include, for example, polyoxyethylene hydrogenated castor oils having 20-100 moles of ethylene oxides, such as polyoxyethylene (20) hydrogenated castor oil, polyethylene (40) hydrogenated castor oil, and polyoxyethylene (100) hydrogenated castor oil.

Polyglycerin alkyl esters having the C10-20 of alkylsubstitute useful herein include, for example, those having 6-10 moles of glycerin units, such as polyglyceryl-6 laurate, polyglyceryl-10 laurate, and polyglyceryl-10 stearate.

Polysorbates useful herein include, for example, those having 20-80 moles of ethylene oxides, such as polysorbate-20, polyborbate-40, polysorbate-60, and polysorbate-80.

Polyethylene sterols and polyethylene hydrogenated sterols useful herein include, for example, those having 10-30 moles of ethylene oxides, such as polyethylene (10) phytosterol, polyethylene (30) phytosterol, and polyethylene (20) cholesterol.

Among the above nonionic surfactants, preferred are polysorbates, and more preferred are polysorbate-20, polysorbate-40, and mixtures thereof.

### Aqueous Carrier

The compositions of the present invention comprise an aqueous carrier for providing the continuous phase. The level and species of the carrier are selected according to the compatibility with other components, and other desired characteristic of the product. The aqueous carrier is contained in the composition at a level by weight of, preferably from about 30% to about 98%, more preferably from about 50% to about 95%, still more preferably from about 70% to about 95%.

Carriers useful in the present invention include water and water solutions of lower alkyl alcohols. Lower alkyl alcohols useful herein are monohydric alcohols having 1 to 6 carbons, more preferably ethanol. Preferably, the aqueous carrier is substantially water. Deionized water is preferably used. Water from natural sources including mineral cations can also be used, depending on the desired characteristic of the product.

The pH of the present composition is preferably from about 4 to about 8. When skin benefit agents are included in the liquid composition, the pH may be adjusted to that which provides optimum efficacy of the active skin benefit agents. Buffers and other pH adjusting agents can be included to achieve the desirable pH. Suitable pH adjusters herein include acetates, phosphates, citrates, triethanolamines and carbonates.

### Viscosifying Agent

The present composition may comprise a viscosifying agent that provides the composition a viscosity of from about 500 mPas to about 1,000,000 mPas, preferably from about 1000 mPas to about 100,000 mPas. The present composition may have a wide range of viscosity. The term “water soluble or water miscible” with regard to the viscosifying agents herein, relate to compounds that are dissolved to make a transparent solution when dissolved in ample amount of water with or without the aid of elevated temperature and/or mixing.

Useful herein are carboxylic acid/carboxylate copolymers. The carboxylic acid/carboxylate copolymer is particularly useful for providing microemulsions as the copolymer keeps the composition relatively transparent and at a suitable viscosity without making the composition tacky or greasy upon use. Additionally, the carboxylic acid/carboxylate copolymer is capable of dispersing and stabilizing water insoluble components in the present composition when such component is included.

Commercially available carboxylic acid/carboxylate copolymers useful herein include: CTFA name Acrylates/C10-30 Alkyl Acrylate Crosspolymer having tradenames Pemulen TR-1, Pemulen TR-2, Carbopol 1342, Carbopol 1382, and Carbopol ETD 2020, all available from B. F. Goodrich Company.

Neutralizing agents may be included to neutralize the carboxylic acid/carboxylate copolymers herein. Nonlimiting examples of such neutralizing agents include sodium hydroxide, potassium hydroxide, ammonium hydroxide, monoethanolamine, diethanolamine, triethanolamine, diisopropanolamine, aminomethylpropanol, tromethamine, tetrahydroxypropyl ethylenediamine, and mixtures thereof.

Useful herein are cellulose derivative polymers. Cellulose derivative polymers useful herein include methylcellulose, ethylcellulose, hydroxyethylcellulose, hydroxyethyl ethylcellulose, hydroxypropyl methyl cellulose, nitrocellulose, sodium cellulose sulfate, sodium carboxymethylcellulose, crystalline cellulose, cellulose powder, and mixtures thereof. Particularly preferred are hydroxyethylcellulose carboxymethylcellulose, and mixtures thereof. Commercially available compounds that are highly useful herein include hydroxyethylcellulose with tradename Natrosol Hydroxyethylcellulose, and carboxymethylcellulose with tradename Aqualon Cellulose Gum, both available from Aqualon.

Useful herein are vinyl polymers such as cross linked acrylic acid polymers with the CTFA name Carbomer, pullulan, mannan, scleroglucans, polyvinylpyrrolidone, polyvinyl alcohol, guar gum, hydroxypropyl guar gum, xanthan gum, acacia gum, arabia gum, tragacanth, galactan, carob gum, karaya gum, locust bean gum, carrageenin, pectin, amylopectin, agar, quince seed (Cydonia oblonga Mill), starch (rice, corn, potato, wheat), algae colloids (algae extract), microbiological polymers such as dextran, succinoglucan, starch-based polymers such as carboxymethyl starch, methylhydroxypropyl starch, alginic acid-based polymers such as sodium alginate, alginic acid propylene glycol esters, acrylate polymers such as sodium polyacrylate, polyacrylamide, polyethyleneimine, and inorganic water soluble material such as bentonite, aluminum magnesium silicate, laponite, hectonite, and anhydrous silicic acid.

Polyalkylene glycols having a molecular weight of more than about 1000 are useful herein. Useful are those having the following general formula: 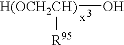
wherein R⁹⁵ is selected from the group consisting of H, methyl, and mixtures thereof. When R⁹⁵ is H, these materials are polymers of ethylene oxide, which are also known as polyethylene oxides, polyoxyethylenes, and polyethylene glycols. When R⁹⁵ is methyl, these materials are polymers of propylene oxide, which are also known as polypropylene oxides, polyoxypropylenes, and polypropylene glycols. When R⁹⁵ is methyl, it is also understood that various positional isomers of the resulting polymers can exist. In the above structure, x3 has an average value of from about 1500 to about 25,000, preferably from about 2500 to about 20,000, and more preferably from about 3500 to about 15,000. Other useful polymers include the polypropylene glycols and mixed polyethylene-polypropylene glycols, or polyoxyethylene-polyoxypropylene copolymer polymers. Polyethylene glycol polymers useful herein are PEG-2M wherein R⁹⁵ equals H and x3 has an average value of about 2,000 (PEG-2M is also known as Polyox WSR® N-10, which is available from Union Carbide and as PEG-2,000); PEG-5M wherein R⁹⁵ equals H and x3 has an average value of about 5,000 (PEG-5M is also known as Polyox WSR® N-35 and Polyox WSR® N-80, both available from Union Carbide and as PEG-5,000 and Polyethylene Glycol 300,000); PEG-7M wherein R⁹⁵ equals H and x3 has an average value of about 7,000 (PEG-7M is also known as Polyox WSR® N-750 available from Union Carbide); PEG-9M wherein R⁹⁵ equals H and x3 has an average value of about 9,000 (PEG 9-M is also known as Polyox WSR® N-3333 available from Union Carbide); and PEG-14 M wherein R⁹⁵ equals H and x3 has an average value of about 14,000 (PEG-14M is also known as POLYOX WSR® N-3000 available from Union Carbide).

Commercially available additional water soluble polymers highly useful herein include xanthan gum with tradename KELTROL series available from Kelco, Carbomers with tradenames CARBOPOL 934, CARBOPOL 940, CARBOPOL 950, CARBOPOL 980, and CARBOPOL 981, all available from B. F. Goodrich Company, acrylates/steareth-20 methacrylate copolymer with tradename ACRYSOL 22 available from Rohm and Hass, polyacrylamide with tradename SEPIGEL 305 available from Seppic, glyceryl polymethacrylate with tradename LUBRAGEL NP, and a mixture of glyceryl polymethacrylate, propylene glycol and PVM/MA copolymer with tradename LUBRAGEL OIL available from ISP, scleroglucan with tradename Clearogel SC11 available from Michel Mercier Products Inc. (NJ, USA), ethylene oxide and/or propylene oxide based polymers with tradenames CARBOWAX PEGs, POLYOX WASRs, and UCON FLUIDS, all supplied by Amerchol.

Useful herein are commercially available amphoteric polymers such as Polyquaternium 22 with tradenames MERQUAT 280, MERQUAT 295, Polyquaternium 39 with tradenames MERQUAT PLUS 3330, MERQUAT PLUS 3331, and Polyquaternium 47 with tradenames MERQUAT 2001, MERQUAT 2001N, all available from Calgon Corporation.

### Water Soluble Humectant

The composition of the present invention may further comprise from about 0.1% to about 30%, preferably from about 3% to about 15% of a water soluble humectant. Water soluble humectants useful herein include polyhydric alcohols such as butylene glycol (1,3 butanediol), pentylene glycol (1,2-pentanediol), glycerin, sorbitol, propylene glycol, hexylene glycol, ethoxylated glucose, 1,2-hexane diol, 1,2-pentane diol, hexanetriol, dipropylene glycol, erythritol, trehalose, diglycerin, xylitol, maltitol, maltose, glucose, fructose; and other water-soluble compounds such as urea, sodium chondroitin sulfate, sodium hyaluronate, sodium adenosin phosphate, sodium lactate, pyrrolidone carbonate, glucosamine, cyclodextrin, and mixtures thereof. Also useful herein include water soluble alkoxylated nonionic polymers such as polyethylene glycols and polypropylene glycols having a molecular weight of up to about 1000 such as those with CTFA names PEG-200, PEG-400, PEG-600, PEG-1000, and mixtures thereof.

Commercially available humectants herein include: butylene glycol with tradename 1,3-Butylene glycol available from Celanese, pentylene glycol with tradename HYDROLITE-5 available from Dragoco, glycerin with tradenames STAR and SUPEROL available from The Procter & Gamble Company, CRODEROL GA7000 available from Croda Universal Ltd., PRECERIN series available from Unichema, and a same tradename as the chemical name available from NOF; propylene glycol with tradename LEXOL PG-865/855 available from Inolex, 1,2-PROPYLENE GLYCOL USP available from BASF; sorbitol with tradenames LIPONIC series available from Lipo, SORBO, ALEX, A-625, and A-641 available from ICI, and UNISWEET 70, UNISWEET CONC available from UPI; dipropylene glycol with the same tradename available from BASF; diglycerin with tradename DIGLYCEROL available from Solvay GmbH; xylitol with the same tradename available from Kyowa and Eizai; maltitol with tradename MALBIT available from Hayashibara, sodium chondroitin sulfate with the same tradename available from Freeman and Bioiberica, and with tradename ATOMERGIC SODIUM CHONDROITIN SULFATE available from Atomergic Chemetals; sodium hyaluronate available from Chisso Corp, the same with tradenames ACTIMOIST available from Active Organics, AVIAN SODIUM HYALURONATE series available from Intergen, HYALURONIC ACID Na available from Ichimaru Pharcos; sodium adenosin phophate with the same tradename available from Asahikasei, Kyowa, and Daiichi Seiyaku; sodium lactate with the same tradename available from Merck, Wako, and Showa Kako, cyclodextrin with tradenames CAVITRON available from American Maize, RHODOCAP series available from Rhone-Poulenc, and DEXPEARL available from Tomen; polyethylene glycols with the tradename CARBOWAX series available from Union Carbide, and a mixture of glyceryl polymethacrylate, propylene glycol and PVM/MA copolymer with tradename Lubrajel Oil available from Guardian Lab.

### Skin Tone Changing Agent

The compositions of the present invention may include a skin tone changing agent as a skin benefit agent. The skin tone changing agent useful herein are selected from the group consisting of skin tone changing pigments, reflective particulate material, and mixtures thereof. Skin tone changing agents useful herein are those altering the appearance of the color and/or tone of the skin including, but not limited to, skin whitening. The skin tone changing agents have a particle size of, preferably at least about 100 nm.

The skin tone changing pigments useful herein include, for example, talc, mica, silica, magnesium silicate, titanium oxide, zinc oxide, and titanium oxide coated mica.

The reflective particulate materials useful herein have a primary particle size of from about 100 nm to about 10 μm (i.e., in the essentially pure, powder form prior to combination with any carrier). The reflective particulate materials can be inorganic. The inorganic reflective particulate materials useful herein include, for example, titanium dioxide, zinc oxide, more preferably the particles consist essentially of titanium dioxide. The inorganic reflective particulate materials can be coated with a coating material such as cationic polymers, cationic surfactants, anionic polymers, and anionic surfactants.

### Additional Skin Active Agent

The compositions of the present invention may further comprise a safe and effective amount of an additional skin active agent. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% of an additional skin active agent.

The type and amount of skin active agents are selected so that the inclusion of a specific agent does not affect the stability of the composition.

Skin lightening agents useful herein refer to active ingredients that improve hyperpigmentation as compared to pre-treatment. Useful skin lightening agents herein include ascorbic acid compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C50 available from DSM.

Other hydrophobic skin lightening agents useful herein include ascorbic acid derivatives such as ascorbyl tetraisopalmitate (for example, VC-IP available from Nikko Chemical), ascorbyl palmitate (for example available from DSM Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Other skin active agents useful herein include those selected from the group consisting of panthenol, benzoyl peroxide, 3-hydroxy benzoic acid, farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

### UV Absorbing Agent

The compositions of the present invention may further comprise a safe and effective amount of a UV absorbing agent. A wide variety of conventional UV protecting agents are suitable for use herein, such as those decribed in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

### Additional Components

The compositions hereof may further contain additional components such as are conventionally used in topical products, e.g., for providing aesthetic or functional benefit to the composition or skin, such as sensory benefits relating to appearance, smell, or feel, therapeutic benefits, or prophylactic benefits. It is to be understood that the above-described required materials may themselves provide such benefits.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

### Examples 1-8

Compositions

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Niacinamide *1 | 3.5 | 5.0 | 3.5 | 5.0 | 3.5 | 5.0 | 2.0 | 3.5 |
| Glucosyl Hesperidin *2 | 0.2 | | 0.1 | 0.1 | 0.2 | 0.2 | 0.1 | |
| Rutin *3 | | 0.2 | 0.1 | 0.1 | | | | 0.2 |
| Pyridoxine Palmitate *4 | 0.2 | 0.2 | 0.1 | 0.2 | 0.2 | 0.2 | 0.1 | 0.2 |
| Octyl Methoxycinnamate | | | | | | | 6.0 | 3.0 |
| Homosalate | | | | | | | | 8.0 |
| Phenylbenzimidazole Sulfonic Acid | | | | | | | | 2.0 |
| Glycerin | 7.0 | 7.0 | 5.0 | 7.0 | | | 6.0 | 4.0 |
| Butylene Glycol | | | 3.0 | | 3.0 | 5.0 | | 3.0 |
| Isohexadecane | 2.0 | 2.0 | 1.0 | 2.0 | 2.0 | 2.0 | 2.0 | |
| Isopropyl Palmitate | 2.0 | 2.0 | 1.0 | 2.0 | | | 2.0 | 1.5 |
| Sucrose Polycottonseedate *5 | 0.5 | 0.5 | 0.2 | 0.5 | | | 0.5 | 0.5 |
| Polyacrylamide *6 | 1.5 | 2.25 | 1.0 | 2.0 | | | 1.6 | 3.5 |
| Acrylates/C10-30 Alkyl | | | | | 0.4 | 0.4 | | |
| Acrylates Crosspolymer *7 | | | | | | | | |
| Xanthan Gum *8 | | | | | 0.1 | 0.1 | | 0.1 |
| Polysorbate 20 | | | | | 0.2 | 0.2 | | |
| Steareth-21 | | | | | | | 0.9 | 0.2 |
| Cetearyl Glucoside *9 | 0.2 | 0.2 | 0.2 | 0.2 | | | 0.2 | 0.2 |
| PEG-100 Stearate *10 | 0.1 | 0.1 | 0.1 | 0.1 | | | | 0.1 |
| Behenyl Alcohol | 0.4 | 0.4 | 0.2 | 0.4 | | | 0.8 | 0.4 |
| Stearyl Alcohol | 0.48 | 0.6 | 0.3 | 0.5 | | | 0.8 | 0.4 |
| Cetyl Alcohol | 0.32 | 0.5 | 0.2 | 0.3 | | | 0.8 | 0.3 |
| Dimethicone and Dimethiconol *11 | 2.0 | 2.0 | 0.5 | 2.0 | 1.5 | 1.5 | 0.8 | 2.0 |
| Tocopheryl Acetate *12 | 0.2 | 0.2 | 0.2 | 0.2 | | 0.2 | 0.2 | 0.2 |
| Panthenol *13 | 0.5 | 0.5 | 0.5 | 0.5 | | 0.5 | 0.5 | 0.5 |
| Sodium Ascorbyl Phosphate *14 | 0.5 | 0.2 | | 0.5 | 2.0 | | | 0.5 |
| Mulberry Extract *15 | 0.5 | 0.5 | 0.2 | 0.5 | 0.5 | | | |
| Ginkgo Extract *16 | 0.1 | 0.1 | 0.1 | 0.1 | 0.2 | 0.5 | | |
| Promatrixyl *17 | | 0.3 | | | | 0.3 | | |
| Disodium EDTA | 0.1 | 0.1 | 0.1 | 0.1 | 0.1 | 0.1 | 0.1 | 0.1 |
| Polymethylsilesquioxane *18 | | | | | 4.0 | 4.0 | | |
| Zinc Oxide *19 | | | | | | | 3.0 | |
| Titanium Dioxide *20 | 0.4 | 0.4 | | | 0.3 | | 0.8 | 0.4 |
| Titanium Dioxide covered | | | | 0.8 | | | 0.5 | |
| Mica *21 | | | | | | | | |
| Polyethylene *22 | | | | | | | 1.0 | 1.0 |
| Ethylparaben | 0.2 | 0.2 | 0.2 | 0.2 | | | 0.2 | 0.2 |
| Methylparaben | 0.2 | 0.2 | 0.2 | 0.2 | 0.1 | 0.1 | 0.25 | 0.25 |
| Propylparaben | 0.1 | 0.1 | 0.1 | 0.1 | | | 0.15 | 0.15 |
| Benzyl Alcohol | 0.5 | 0.5 | 0.5 | 0.5 | 0.2 | 0.2 | 0.5 | 0.5 |
| Saccharomycopsis Ferment | | | | | | 30 | | |
| Filtrate *23 | | | | | | | | |
| Perfume | 0.2 | 0.2 | 0.1 | 0.2 | 0.1 | 0.1 | 0.2 | 0.2 |

| | |
|---|---|
| Sodium Hydroxide | Adjust to pH = 5.5-7.5 |
| Water | Make total 100 |

Definitions of Components
*1 Niacinamide: Niacinamide USP available from DSM
*2 Glucosyl Hesperidin: alpha-Ghesperidin PS-CC, available from Toyo Sugar Refining.
*3 Rutin: available from Alps Pharmaceutical Industry
*4 Pyridoxine Palmitate: Nikkol DP, available from Nikko Chemicals.
*5 Sucrose Polycottonseedate: available from Kobo Products Inc.
*6 Polyacrylamide: Sepigel 305 available from SEPPIC Inc.
*7 Acrylates/C10-30 Alkyl Acrylates Crosspolymer: Pemulen TR-2 available from B. F. Goodrich
*8 Xanthan Gum: Keltrol available from Kelco
*9 Cetearyl Glucoside: Emulgade PL 68/50, available from Cognis
*10 PEG-100 Stearate: Myrj 59P, available from Uniqema
*11 Dimethicone and Dimethiconol: DC Q2-1503 fluid available from Dow Chemicals.
*12 Tocopheryl Acetate: Available from Eisai Co. Ltd.
*13 Panthenol: D-Panthenol available from DSM
*14 Sodium Ascorbyl Phosphate: Stay-C 50 available from DSM
*15 Mulberry Extract: Mulberry extract BG available from Maruzen Pharmaceuticals
*16 Ginkgo Extract: Ginkgo Extract BG, available from Maruzen Pharmaceuticals
*17 Promatrixyl: mixture contains Palmitoyl Pentapeptide-3, Available Sederma, Inc.
*18 Polymethylsilesquioxane: Tospearl 145A, available from GE Toshiba
*19 Zinc Oxide, Z-Cote HP1, available from BASF Corp.
*20 Titanium Dioxide: Kobo GLW75CAP-MP available from Kobo Products Inc.
*21 Titanium Dioxide covered Mica: Flamenco Blue available from Engelhard.
*22 Polyethylene: Microthene FN 510-00, available from Equistar Chemicals.
*23 Saccharomycopsis Ferment Filtrate: SKII Pitera available from Kashiwayama

### Method of Preparation

The compositions above described can be made by any method known to the artisan. The compositions are suitably made as follows:
(1) Any oily component and hydrophilic surfactants are mixed in a vessel, and heated to 70° C. or above in order to melt solid oily compounds if included. ZnO or other inorganic powder is dispersed into oily phase too, if included.
(2) Water-soluble and heat-stable ingredients other than water soluble thickening polymers are dissolved in water in another vessel, and heated to 70° C. or above.
(3) The products of steps (1) and (2) are mixed with a high speed mixer or mill or homogenizer, mixed until homogeneous.
(4) Water soluble thickening polymers are added to the product of step (3) and mixed until homogeneous.
(5) Polyethylene beads or premixed TiO2, is added into the above mixture, and mixed.
(6) The product of step (5) is cooled down to 40° C. or below.
(7) All other water soluble ingredient, like Niacinamide, glucosyl hesperidin, extract, etc., are dissolved into water, and add to the mixture of step (6).
(8) If included, other remaining components such as perfumes and Saccharomycopsis ferment filtrate are added to the product of step (6).

These embodiments represented by the previous examples are useful as skin care products. When applied to the facial skin, they provide many advantages. For example, they can provide improvement in the areas of skin tone, skin lightening, lightening of skin spots, skin sallowness reduction, and fine wrinkle reduction. Significant improvements in the benefits above are observed when the examples are used daily for a period of at least 4 weeks.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A skin care composition comprising:
(1) from about 0.001% to about 10% of a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
(2) from about 0.01% to about 15% of a vitamin B3 compound; and
(3) a dermatologically acceptable oil-in-water carrier comprising at least about 2% by weight of the entire composition of an oil component.

2. The composition of claim 1 further comprising from 0.001% to about 15% of a vitamin B6 compound.

3. (canceled)

4. The composition of claim 1 wherein the flavonoid compound is glucosyl hesperidin.

5. The composition of claim 1 wherein the carrier comprises:
(a) an oily component;
(b) an emulsifier;
(c) a viscosifying agent; and
(d) water.

6. The composition of claim 5 wherein the viscosifying agent comprises an acrylic polymer.

7. The composition of claim 5 further comprising a skin tone changing agent.

8. The composition of claim 1 wherein the carrier comprises:
(a) an emulsifier comprising two or more nonionic surfactants selected from the group consisting of polyoxyalkylene alkyl ether having the C₁₂₋₁₈ of alkyl substitute, polyoxyalkylene hydrogenerated castor oil, and a liner or branched, mono- or tri-alkyl glyceride;
(b) an oily component selected from the group consisting of hydrocarbon oils, fatty acid esters, silicone oils, and mixtures thereof;
(c) a viscosifying agent comprising carboxylic acid/carboxylate copolymer;
(d) a water soluble humectant; and
(e) water,
wherein the composition has an absorbent value of less than about 2 at a wave length of 340 nm.

9. The composition of claim 8, wherein at least one of the nonionic surfactants has an HLB of more than 10 and at least one of the nonionic surfactants has an HLB of less than 10.

10. A method of lightening the skin comprising the step of applying to the skin a composition comprising:
(1) from about 0.001% to about 10% of a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
(2) from about 0.01% to about 15% of a vitamin B3 compound; and
(3) a dermatologically acceptable oil-in-water carrier comprising at least about 2% by weight of the entire composition of an oil component.

11. A method of providing anti-aging benefit to the skin comprising the step of applying to the skin a composition comprising:
(1) from about 0.001% to about 10% of a flavonoid compound selected from the group consisting of glucosyl hesperidin, glucosyl rutin, glucosyl myricitrin, glucosyl isoquercitrin, glucosyl quercitrin, methyl hesperidin, and mixtures thereof;
(2) from about 0.01% to about 15% of a vitamin B3 compound; and
(3) dermatologically acceptable oil-in-water carrier comprising at least about 2% by weight of the entire composition of an oil component.
